(19) United States
(12) Patent Application Publication
(10) Pub. No.: US 2025/0001097 A1
(43) Pub. Date: Jan. 2, 2025

(54) PUNCTURE NEEDLE

(71) Applicant: TERUMO KABUSHIKI KAISHA, Tokyo (JP)

(72) Inventors: Masahiko NAGASAWA, Kai-shi (JP); Kosuke OSAWA, Ebina-shi (JP); Toshiya ASAI, Fuji-shi (JP)

(73) Assignee: TERUMO KABUSHIKI KAISHA, Tokyo (JP)

(21) Appl. No.: 18/882,237

(22) Filed: Sep. 11, 2024

(30) Foreign Application Priority Data
Mar. 15, 2022 (JP) 2022-040680

(51) Int. Cl. A61M 5/32; A61M 5/158

(52) U.S. Cl. CPC A61M 5/3286; A61M 5/158

(57) ABSTRACT

A puncture needle includes: a rod-shaped main body; a blade surface portion formed at a distal end of the main body, the blade surface portion including a front-side blade surface extending so as to be inclined with respect to an axis of the main body; a planar first back surface portion formed on a back surface side of the front-side blade surface at the distal end of the main body; and a planar second back surface portion formed on the back surface side of the front-side blade surface at the distal end of the main body. The first back surface portion and the second back surface portion are parallel to the axis or come closer to each other toward a needle tip, and do not intersect with each other at the needle tip.

### CROSS-REFERENCE TO RELATED APPLICATIONS

This is a bypass continuation of PCT Application No. PCT/JP2023/008913, filed on Mar. 8, 2023, which claims priority to Japanese Patent Application No. JP2022-040680, filed on Mar. 15, 2022. The entire contents of these applications are incorporated by reference herein.

### BACKGROUND

The present disclosure relates to a puncture needle.

WO 2017/017935 A discloses a puncture needle for medical use. The puncture needle is provided with a distal end including a needle tip, and a rod-shaped main body continuous to the distal end. The distal end is provided with a blade surface. The blade surface is provided with a front-surface-side blade surface and a back-surface-side blade surface formed on a back side of the front-surface-side blade surface, and is subjected to back cut processing. The front-surface-side blade surface is provided with a first blade surface portion and a second blade surface portion that form a blade edge including a needle tip at one end by a ridge line at which the first blade surface portion and the second blade surface portion intersect with each other, and a third blade surface portion continuous to the first blade surface portion and the second blade surface portion on the main body side.

### SUMMARY

One of functions of the blade surface portion in the puncture needle is to cut and open the skin, tissue, and the like with a cutting edge. As a result, it is assumed that resistance when inserting a junction, a jaw, and the like of the puncture needle into the skin, tissue, and the like, and resistance when passing a mantle tube can be reduced. However, there has been a case in which the cutting edge of the conventional puncture needle cannot be sharpened, or the length of the cutting edge cannot be made sufficient, and the skin and tissue cannot be smoothly cut and opened, so that insertion resistance increases. Therefore, a puncture needle including a sharp cutting edge with a sufficiently secured length is desired.

The present disclosure has been made in view of such circumstances, and an object thereof is to provide a puncture needle including a sharp cutting edge with a sufficiently secured length.

A puncture needle according to one embodiment of the present disclosure includes: a rod-shaped main body; a blade surface portion formed at a distal end of the main body, the blade surface portion including a front-side blade surface extending so as to be inclined with respect to an axis of the main body; a planar first back surface portion formed on a back surface side of the blade surface at the distal end of the main body; and a planar second back surface portion formed on the back surface side of the blade surface at the distal end of the main body. The first back surface portion and the second back surface portion are parallel to the axis or come closer toward a needle tip, and do not intersect with each other at the needle tip.

According to one aspect of the puncture needle according to the present disclosure, the first back surface portion intersects with the front-side blade surface to form a first side blade edge, the second back surface portion intersects with the front-side blade surface to form a second side blade edge, and the first side blade edge and the second side blade edge come closer to each other toward the needle tip.

According to one aspect of the puncture needle according to the present disclosure, the first side blade edge and the second side blade edge may be formed linearly.

According to one aspect of the puncture needle according to the present disclosure, the blade surface portion may include a first blade surface portion, a second blade surface portion, and a third blade surface portion as the front-side blade surface, the third blade surface portion may be arranged on a proximal end side of the first blade surface portion or the second blade surface portion, the first blade surface portion and the second blade surface portion may intersect with each other to form a needle tip front-side blade edge including the needle tip at one end, the first back surface portion and the first blade surface portion may intersect with each other to form the first side blade edge, and the second back surface portion and the second blade surface portion may intersect with each other to form the second side blade edge.

According to one aspect of the puncture needle according to the present disclosure, the blade surface portion may include a third blade surface portion and a fourth blade surface portion as the front-side blade surface, the fourth blade surface portion may be arranged on a distal end side with respect to the third blade surface portion, and may be significantly inclined with respect to the axis as compared with the third blade surface portion, and the first side blade edge and the second side blade edge may extend from the third blade surface portion to the fourth blade surface portion.

According to one aspect of the puncture needle according to the present disclosure, the blade surface portion may include a third blade surface portion and a fifth blade surface portion, the fifth blade surface portion may be arranged on a distal end side with respect to the third blade surface portion, and may be parallel to or slightly inclined with respect to the axis as compared with the third blade surface portion, and the first side blade edge and the second side blade edge may extend from the third blade surface portion to the fifth blade surface portion.

According to one aspect of the puncture needle according to the present disclosure, the puncture needle further includes: a back-surface-side blade surface formed on the back surface side of the front-side blade surface in the blade surface portion. The back-surface-side blade surface may include: a first back-surface-side blade surface portion formed in a planar shape, the first back-surface-side blade surface portion intersecting with the front-side blade surface to form a third side blade edge, and a second back-surface-side blade surface portion formed in a planar shape, the second back-surface-side blade surface portion intersecting with the front-side blade surface to form a fourth side blade edge, and the first back-surface-side blade surface portion and the second back-surface-side blade surface portion intersect with each other to form a needle tip back-surface-side blade edge including the needle tip at one end.

According to one aspect of the puncture needle according to the present disclosure, the main body is tubular and includes a circular arc-shaped portion having a circular arc-shaped cross section intersecting with the axis, and planar first plate-shaped portion and second plate-shaped portion, the first back surface portion is an outer surface of the first plate-shaped portion, and the second back surface portion is an outer surface of the second plate-shaped portion.

According to certain embodiments of the present disclosure, it is possible to provide a puncture needle including a sharp cutting edge with a sufficiently secured length.

### BRIEF DESCRIPTION OF DRAWINGS

FIG. 1 is a perspective view of a puncture needle according to a first embodiment;

FIG. 2 is a side view of the puncture needle according to the first embodiment;

FIG. 3 is a top view of the puncture needle according to the first embodiment;

FIG. 4 is a back view of the puncture needle according to the first embodiment;

FIG. 5 is a cross-sectional view taken along line V-V of FIG. 3;

FIG. 6 is a cross-sectional view taken along line VI-VI of FIG. 3;

FIG. 7 is a perspective view of a puncture needle according to a second embodiment;

FIG. 8 is a side view of the puncture needle according to the second embodiment;

FIG. 9 is a top view of the puncture needle according to the second embodiment;

FIG. 10 is a cross-sectional view taken along line X-X of FIG. 9;

FIG. 11 is a cross-sectional view taken along line XI-XI of FIG. 9;

FIG. 12 is a cross-sectional view taken along line XII-XII of FIG. 9;

FIG. 13 is a perspective view of a puncture needle according to a third embodiment;

FIG. 14 is a perspective view of a puncture needle according to a fourth embodiment;

FIG. 15 is a side view of the puncture needle according to the fourth embodiment;

FIG. 16 is a top view of the puncture needle according to the fourth embodiment;

FIG. 17 is a side view of a puncture needle according to a fifth embodiment;

FIG. 18 is a top view of the puncture needle according to the fifth embodiment;

FIG. 19 is a back view of the puncture needle according to the fifth embodiment;

FIG. 20 is a cross-sectional view taken along line XX-XX of FIG. 18;

FIG. 21 is a side view of a puncture needle according to a sixth embodiment;

FIG. 22 is a top view of the puncture needle according to the sixth embodiment;

FIG. 23 is a side view of another puncture needle;

FIG. 24 is a perspective view of another puncture needle; and

FIG. 25 is a cross-sectional view taken along line XXV-XXV of FIG. 24.

### DETAILED DESCRIPTION

A puncture needle according to an embodiment of the present disclosure is described with reference to the drawings.

### First Embodiment

### Overview

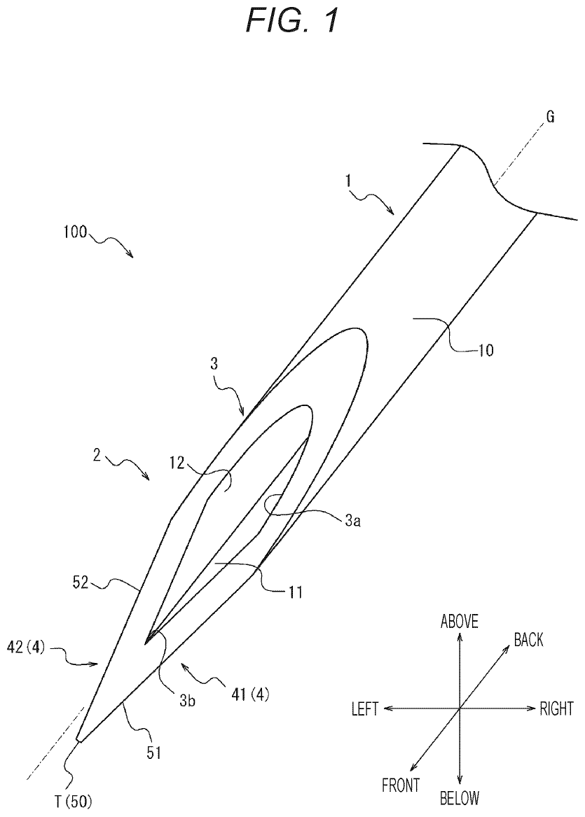

FIGS. 1 to 4 illustrate a puncture needle 100 according to the present embodiment.

The puncture needle 100 is provided with a rod-shaped main body 1, a blade surface portion 2 formed at a distal end of the main body 1 and including a front-side blade surface 3 extending so as to be inclined with respect to an axis G of the main body 1, a planar first back surface portion 41 formed on a back surface side of the front-side blade surface 3 at the distal end of the main body 1, and a planar second back surface portion 42 formed on the back surface side of the front-side blade surface 3 at the distal end of the main body 1.

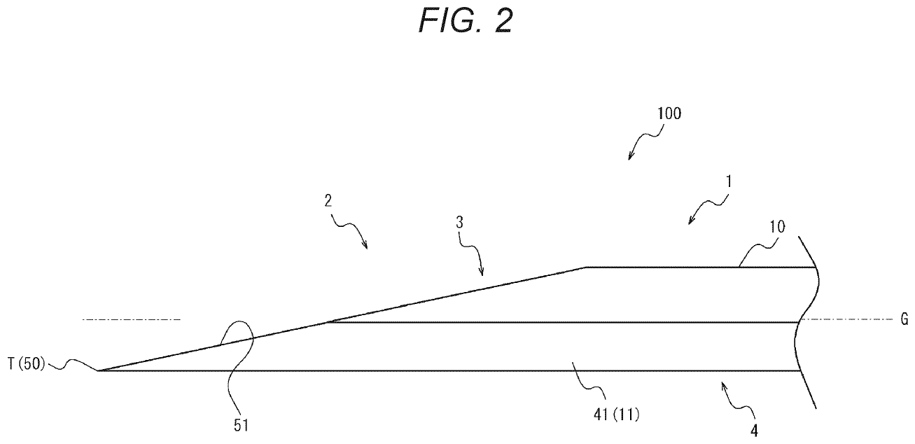

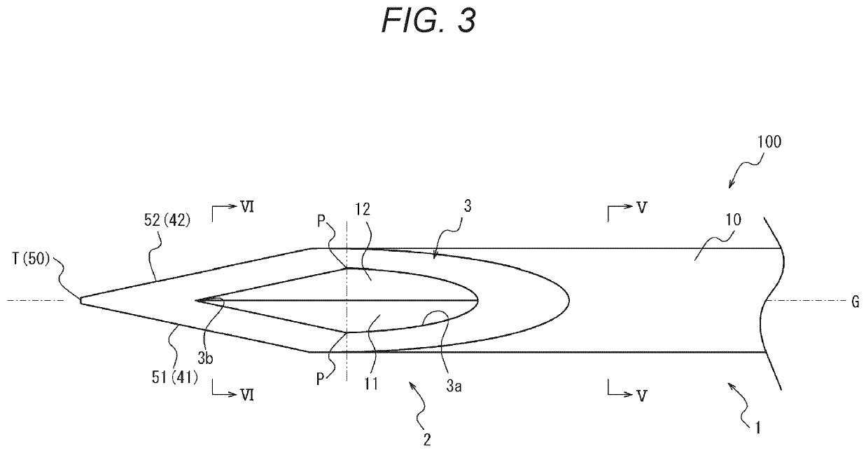

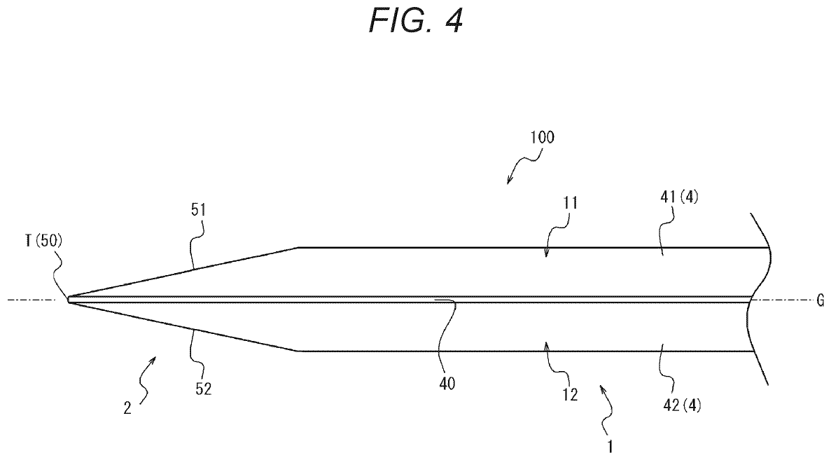

FIG. 1 is a perspective view of the puncture needle 100 as seen obliquely from above the side (front surface side) on which the front-side blade surface 3 is formed. FIG. 2 is a view as seen in a direction intersecting with the axis G, a side view of the puncture needle 100 when the front-side blade surface 3 is seen from the side. FIG. 3 is a view as seen in a direction intersecting with the axis G, a top view of the puncture needle 100 as seen from the front surface side on which the front-side blade surface 3 is formed. FIG. 4 is a view as seen in a direction intersecting with the axis G, a bottom view of the puncture needle 100 as seen from the back surface side on a side opposite to the front surface side on which the front-side blade surface 3 is formed.

In the following description, as illustrated in FIG. 1, in the puncture needle 100, a side on which a jaw of the front-side blade surface 3 is present in the direction intersecting with the axis G is referred to as above, an upper side, front, or a front side, and the opposite side is referred to as below, a lower side, a back surface, or a back surface side. A side on which a needle tip T, which is a distal end of the puncture needle 100, is present in the direction along the axis G is referred to as the front or a distal end side, and the opposite side is referred to as the back or a proximal end side. A left-hand side and a right-hand side are sometimes referred to as left and right, respectively, when the puncture needle 100 is seen in the direction along the axis G.

The first back surface portion 41 and the second back surface portion 42 are parallel to the axis G, or come closer to each other toward the needle tip T and do not intersect with each other at the needle tip T. As a result, it is possible to provide the puncture needle 100 in which a first side blade edge 51 and a second side blade edge 52 as cutting edges are sharp, and lengths of the first side blade edge 51 and the second side blade edge 52 are sufficiently secured.

### Detailed Explanation

The puncture needle 100 illustrated in FIGS. 1 to 4 is, for example, a puncture needle for medical use. The puncture needle 100 is provided with the main body 1, the blade surface portion 2 including the front-side blade surface 3, the first back surface portion 41, and the second back surface portion 42. As illustrated in FIGS. 1 and 3, the blade surface portion 2, the first back surface portion 41, and the second back surface portion 42 are formed on the main body 1. The puncture needle 100 is bilaterally symmetrical in the present embodiment, but is not limited thereto. Note that, FIG. 1 is the perspective view as seen obliquely from an upper right side, FIG. 2 is a right side view, and FIG. 3 is the top view. FIG. 4 is the bottom view as seen from a side opposite to FIG. 3 by 180 degrees.

As illustrated in FIG. 1, the main body 1 is formed into a tubular shape as an example of a rod shape. The main body 1 is formed of, for example, a metal alloy such as stainless steel, a titanium alloy, or a cobalt-chromium alloy, or a fluororesin such as polytetrafluoroethylene, or a resin such as nylon.

In the following description, an outer surface of a tube in the tubular main body 1, that is, an outer surface in a radial direction around the axis G is sometimes collectively referred to as an outer surface. An inner surface of the tube in the tubular main body 1, that is, an inner surface in the radial direction around the axis G is sometimes collectively referred to as an inner surface.

As illustrated in FIGS. 1 to 4, the axis G is an axis that overlaps the center of gravity of a cross section in an extending direction of the main body 1 and extends in the extending direction of the main body 1. The inside of the tube of the main body 1 serves as a flow path through which a liquid such as a drug solution or a biological fluid of a patient flows. The proximal end side of the main body 1 may be connected to, for example, a syringe for injection or a tube that allows the drug solution or the biological fluid to flow. In a case in which the tube is connected to the proximal end side of the main body 1, a hub can be provided on the proximal end side of the main body 1, and the tube can be connected via the hub.

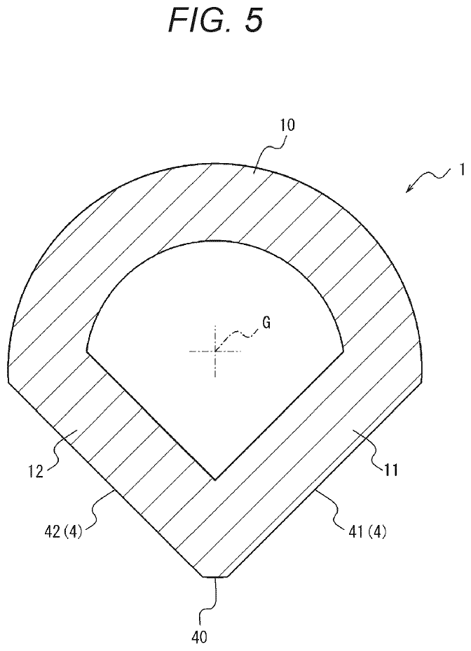

FIG. 5 is a cross-sectional view taken along line V-V in FIG. 3. The main body 1 includes a circular arc-shaped portion 10 having a circular arc-shaped cross section intersecting with the axis G, and a first plate-shaped portion 11 and a second plate-shaped portion 12 each having a planar shape. The circular arc-shaped portion 10 is arranged on the front side. The circular arc-shaped portion 10 has a semicircular shape in the present embodiment. The first plate-shaped portion 11 and the second plate-shaped portion 12 are arranged on the back surface side. The first plate-shaped portion 11 is arranged on a right side. The second plate-shaped portion 12 is arranged on a left side. An upper end of the first plate-shaped portion 11 is continuous to a lower end on the right side of the circular arc-shaped portion 10. An upper end of the second plate-shaped portion 12 is continuous to a lower end on the left side of the circular arc-shaped portion 10. Lower ends of the first plate-shaped portion 11 and the second plate-shaped portion 12 are continuous to each other. The first plate-shaped portion 11 and the second plate-shaped portion 12 form a cross section intersecting with the axis G in a substantially V shape of an alphabet. In this manner, the shape of the cross section intersecting with the axis G of the main body 1 is a circular arc shape in which the front surface side is convex upward, and a triangular portion convex downward is added below the circular arc portion.

The lower end at which the first plate-shaped portion 11 and the second plate-shaped portion 12 are continuous to each other, that is, a V-shaped lower end (V-shaped corner portion) formed by the first plate-shaped portion 11 and the second plate-shaped portion 12 is a lower end surface 40 of which outer surface is formed into a chamfered planar shape or curved surface shape.

In the present embodiment, the circular arc-shaped portion 10, the first plate-shaped portion 11, the second plate-shaped portion 12, and the lower end surface 40 extend in parallel with the axis G. It is sufficient that the first plate-shaped portion 11 and the second plate-shaped portion 12 are arranged on a distal end side with respect to the proximal end of the front-side blade surface 3 in the direction along the axis G. It is preferable that the first plate-shaped portion 11 and the second plate-shaped portion 12 extend from the distal end of the main body 1 to the proximal end side with respect to a position at which a width in a right-to-left direction of an opening 3a in the direction along the axis G is maximum. The first plate-shaped portion 11 and the second plate-shaped portion 12 may be formed up to the proximal end of the main body 1.

The main body 1 may be formed into the above-described shape by, for example, forming processing of a cylindrical tubular member by pressing, drawing with a die, bending such as rolling, and the like. In this case, the forming processing of the main body 1 may be performed before the front-side blade surface 3 is formed. For example, the first back surface portion 41 and the second back surface portion 42 may be formed before the front-side blade surface 3 is formed.

As illustrated in FIGS. 1 to 3, the blade surface portion 2 is formed on the distal end side of the main body 1. In other words, the side on which the blade surface portion 2 is formed in the direction along the axis G of the main body 1 is the distal end side of the puncture needle 100. The blade surface portion 2 includes the front-side blade surface 3.

The front-side blade surface 3 is a blade surface that extends from the proximal end side to the distal end side while inclining with respect to the axis G of the main body 1 from an upper side to a lower side. In the present embodiment, the front-side blade surface 3 is formed in one planar shape. As illustrated in FIGS. 1 and 3, the opening 3a communicating with the inside of the tube of the main body 1 is formed in the central portion of the front-side blade surface 3.

As illustrated in FIGS. 4 and 5, the first back surface portion 41 and the second back surface portion 42 are planar surface portions formed on the back surface side. Hereinafter, the first back surface portion 41 and the second back surface portion 42 are sometimes collectively referred to as the back surface portion 4. In the present embodiment, the outer surface of the first plate-shaped portion 11 is the first back surface portion 41. The outer surface of the second plate-shaped portion 12 is the second back surface portion 42.

In the present embodiment, the back surface portion 4, that is, the first back surface portion 41 and the second back surface portion 42 extend in parallel with the axis G. In other words, the first back surface portion 41 and the second back surface portion 42 extend in parallel, do not intersect with each other, and do not form a ridge line. It is sufficient that the back surface portion 4 is arranged on the distal end side with respect to the proximal end of the front-side blade surface 3 in the direction along the axis G. It is preferable that the back surface portion 4 extends from the distal end of the main body 1 to the proximal end side with respect to the position at which the width in the right-to-left direction of the opening 3a in the direction along the axis G is maximum. Note that, the back surface portion 4 is formed at the distal end of the main body 1. The back surface portion 4 may be formed in a range up to the proximal end of the main body 1, but is not necessarily formed in the range up to the proximal end of the main body 1.

As illustrated in FIGS. 1 and 3, the first back surface portion 41 intersects with the front-side blade surface 3 to form a ridge line, and the ridge line forms the first side blade edge 51. The second back surface portion 42 intersects with the front-side blade surface 3 to form a ridge line, and the ridge line forms the second side blade edge 52. The first side blade edge 51 and the second side blade edge 52 come closer to each other toward the needle tip T, but do not merge or intersect with each other on the distal end side. The first side blade edge 51 and the second side blade edge 52 form cutting edges in the blade surface portion 2.

Note that, as illustrated in FIGS. 1 to 4, because the front-side blade surface 3 intersects with the lower end surface 40 at the needle tip T to form a distal end blade edge 50, the first side blade edge 51 and the second side blade edge 52 do not intersect with each other at the needle tip T.

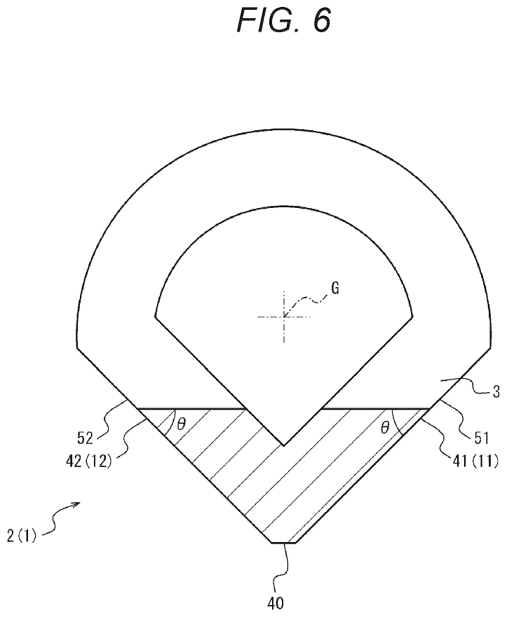

As illustrated in FIGS. 1, 3, and 4, the first side blade edge 51 and the second side blade edge 52 are formed linearly because the front-side blade surface 3, and the first back surface portion 41 and the second back surface portion 42 intersecting with the same are planar. Because the first back surface portion 41 and the second back surface portion 42 are planar, cutting edge angles θ (refer to FIG. 6) of the first side blade edge 51 and the second side blade edge 52 become sharp (for example, cutting edge angle θ=45° as compared with a case in which portions corresponding to the first back surface portion 41 and the second back surface portion 42 have a curved surface shape protruding outward as in a case in which the main body 1 is cylindrical, and as a result, the cutting edge becomes sharp.

Note that, as illustrated in FIG. 3, for example, the first side blade edge 51 and the second side blade edge 52 may be formed on the front-side blade surface 3 from the same position as the position at which the width in the right-to-left direction of the opening 3a in the direction along the axis G is maximum (the position of a virtual line P-P in FIG. 3) or the position slightly closer to the proximal end side toward the distal end side.

As illustrated in FIGS. 3 and 4, the first side blade edge 51 and the second side blade edge 52 can be set to appropriate lengths by widening or narrowing widths in a circumferential direction of the main body 1 of the first back surface portion 41 and the second back surface portion 42. That is, by setting the lengths of the first side blade edge 51 and the second side blade edge 52 to necessary lengths, the puncture needle 100 can secure a necessary and sufficient length of the cutting edge in the blade surface portion 2.

Because the main body 1 includes the circular arc-shaped portion 10 (refer to FIG. 3) having the circular arc-shaped cross section intersecting with the axis G, and the planar first plate-shaped portion 11 and second plate-shaped portion 12 (refer to FIG. 4), the first plate-shaped portion 11 and second plate-shaped portion 12 are arranged on the back surface side, the outer surface of the first plate-shaped portion 11 is the first back surface portion 41, and the outer surface of the second plate-shaped portion 12 is the second back surface portion 42, it is possible to form the first side blade edge 51 and the second side blade edge 52 as the cutting edges while securing sufficiently long lengths as compared with a case in which the main body 1 has a cylindrical shape.

### Second Embodiment

In the first embodiment described above, the case in which the front-side blade surface 3 is the blade surface that extends from the proximal end side to the distal end side while inclining with respect to the axis G of the main body 1 from the upper side to the lower side is described, and the case in which the front-side blade surface 3 is formed in one planar shape is described. However, the front-side blade surface 3 is not always formed in one planar shape. The front-side blade surface 3 may be a combination of two or more (for example, three) planes.

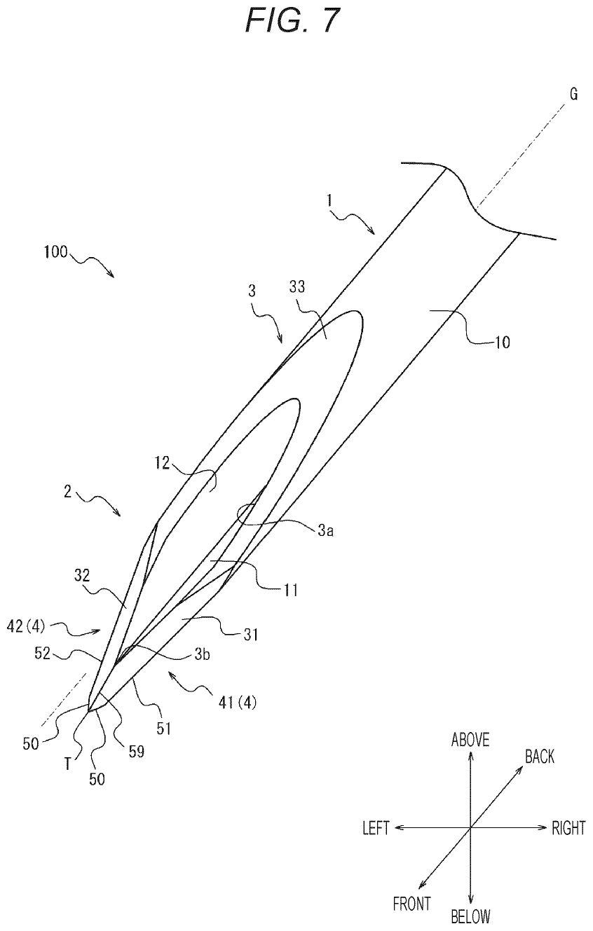

FIGS. 7 to 9 illustrate a puncture needle 100 according to the present embodiment. FIGS. 7 to 9 are a perspective view, a right-side view, and a top view of the puncture needle 100, respectively, similarly to FIGS. 1 to 3. The puncture needle 100 according to the present embodiment is different from the puncture needle 100 of the first embodiment with respect to the front-side blade surface 3, but is otherwise the same as the first embodiment. Hereinafter, portions different from those of the first embodiment are mainly described, and the description of the similar portions is omitted as appropriate.

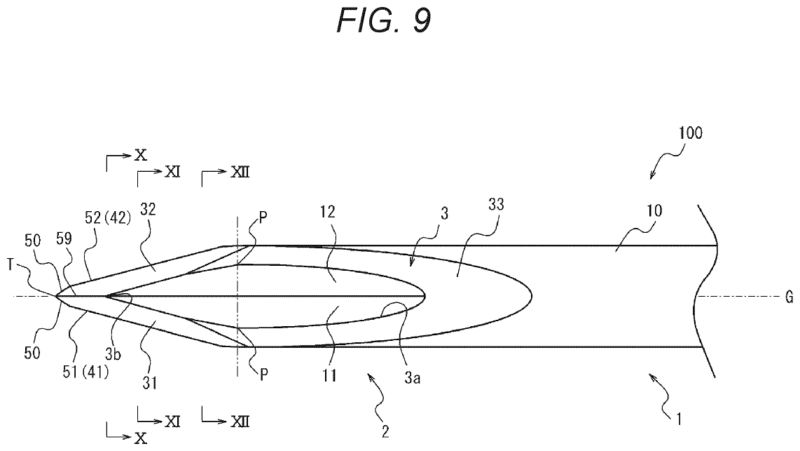

As illustrated in FIGS. 7 and 9, a blade surface portion 2 of the puncture needle 100 according to the present embodiment includes a first blade surface portion 31, a second blade surface portion 32, and a third blade surface portion 33 as a front-side blade surface 3. The third blade surface portion 33 is a surface portion arranged on a proximal end side of the first blade surface portion 31 or the second blade surface portion 32, and is arranged, in the present embodiment, on the proximal end side of the first blade surface portion 31 and the second blade surface portion 32. The puncture needle 100 is a so-called lancet type.

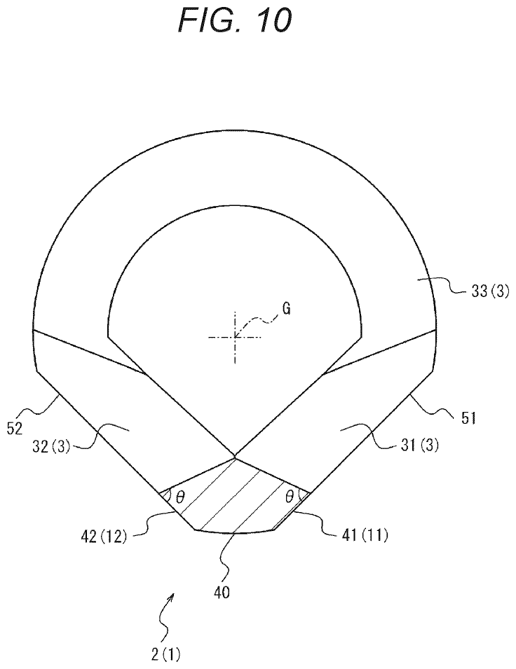

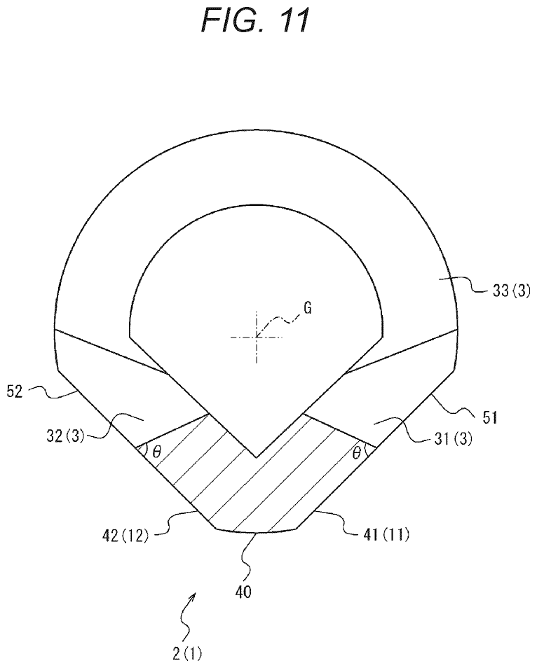

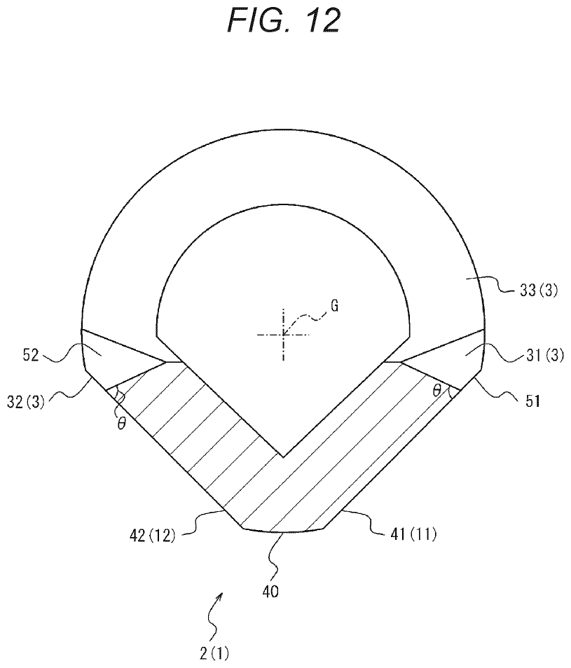

The first blade surface portion 31 and the second blade surface portion 32 are surface portions arranged on a distal end side with respect to the third blade surface portion 33. The first blade surface portion 31 is arranged on a right side of the front-side blade surface 3, and the second blade surface portion 32 is arranged on a left side of the front-side blade surface 3. The first blade surface portion 31 and the second blade surface portion 32 are at the same position in a direction along an axis G. The first blade surface portion 31 and the second blade surface portion 32 are inclined toward an outer side of a tube with respect to the third blade surface portion 33. That is, as illustrated in FIGS. 10 to 12, the first blade surface portion 31 is inclined to the right side. The second blade surface portion 32 is inclined to the left side. As illustrated in FIG. 9, for example, the first blade surface portion 31 and the second blade surface portion 32 may be formed on the front-side blade surface 3 from the same position as the position at which the width in the right-to-left direction of the opening 3a in the direction along the axis G is maximum (the position of a virtual line P-P in FIG. 9) or the position slightly closer to the proximal end side toward the distal end side.

The first blade surface portion 31 and the second blade surface portion 32 intersect with each other at the distal end of the front-side blade surface 3 to form a needle tip front-side blade edge 59 including a needle tip T as one end on the front-side blade surface 3.

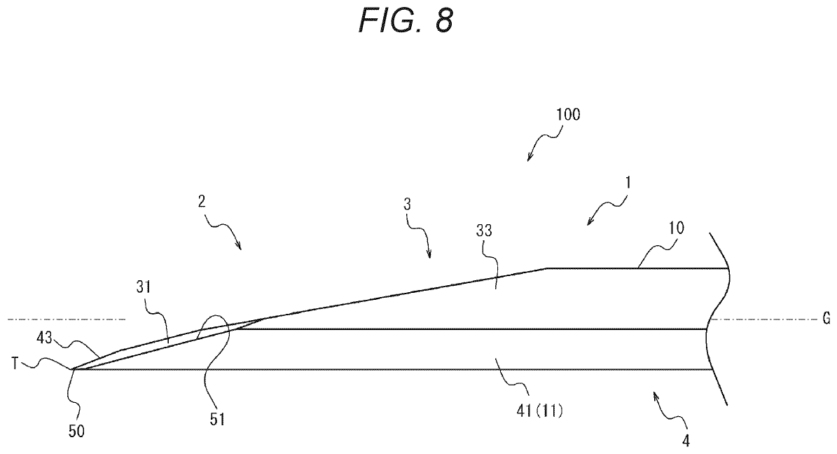

In the puncture needle 100 of the present embodiment, as illustrated in FIGS. 7 and 8, the first back surface portion 41 intersects with the front-side blade surface 3 only with the first blade surface portion 31. The first back surface portion 41 forms a first side blade edge 51 as a cutting edge by a ridge line formed by intersecting with the first blade surface portion 31.

Similarly to the first back surface portion 41, as illustrated in FIG. 7, the second back surface portion 42 intersects with the front-side blade surface 3 only with the second blade surface portion 32. The second back surface portion 42 forms a second side blade edge 52 as a cutting edge by a ridge line formed by intersecting with the second blade surface portion 32.

Note that, as illustrated in FIGS. 7 and 9, because the first blade surface portion 31 and the second blade surface portion 32 of the front-side blade surface 3 intersect with a lower end surface 40 at the needle tip T to form the distal end blade edge 50, the first side blade edge 51 and the second side blade edge 52 do not intersect with each other at the needle tip T.

The first side blade edge 51 and the second side blade edge 52 are formed linearly because the first blade surface portion 31 and the second blade surface portion 32 are planar and the first back surface portion 41 and the second back surface portion 42 intersecting with them are planar. Because the first back surface portion 41 and the second back surface portion 42 are planar, cutting edge angles θ (refer to FIGS. 10 to 12) of the first side blade edge 51 and the second side blade edge 52 become sharp and uniform angles (for example, a constant cutting edge angle θ=70° as compared with a case in which portions corresponding to the first back surface portion 41 and the second back surface portion 42 have a curved surface shape protruding outward from the tube in the main body 1 (for example, the main body 1 has a cylindrical shape), so that the first side blade edge 51 and the second side blade edge 52 as the cutting edges can be made sharp cutting edges with uniform angles. Note that, FIGS. 10 to 12 are cross-sectional views taken along line X-X, line XI-XI, and line XII-XII in FIG. 9, respectively.

The first side blade edge 51 and the second side blade edge 52 can be set to appropriate lengths by widening or narrowing widths in a circumferential direction of the main body 1 of the first back surface portion 41 and the second back surface portion 42, or lengthening or shortening extending lengths in the direction along the axis G of the first blade surface portion 31 and the second blade surface portion 32. That is, by setting the lengths of the first side blade edge 51 and the second side blade edge 52 to necessary lengths, the puncture needle 100 can secure a necessary and sufficient length of the cutting edge in the blade surface portion 2.

### Third Embodiment

In the second embodiment described above, the case in which the first blade surface portion 31 and the second blade surface portion 32 are formed, for example, on the front-side blade surface 3 from the same position as the position at which the width in the right-to-left direction of the opening 3a in the direction along the axis G is maximum (the position of a virtual line P-P in FIG. 9) or the position slightly closer to the proximal end side toward the distal end side; however, arrangement of the first blade surface portion 31 and the second blade surface portion 32 is not limited thereto. A puncture needle 100 of a third embodiment is the same as that of the first embodiment and the second embodiment except for the arrangement of the first blade surface portion 31 and the second blade surface portion 32. Hereinafter, while describing differences from the second embodiment, common points with the first embodiment are mentioned as necessary.

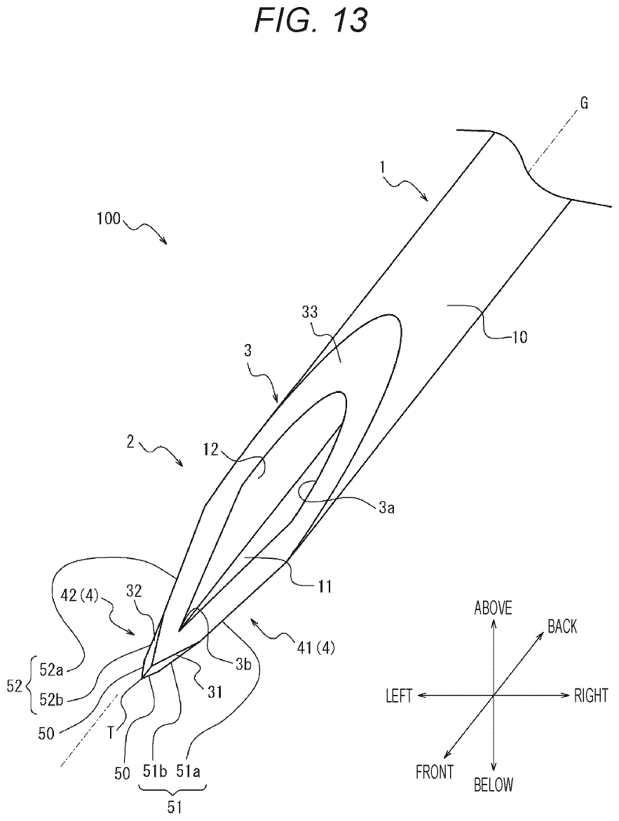

In the present embodiment, the first blade surface portion 31 and the second blade surface portion 32 may be formed from a position on a distal end side with respect to a position at which the width in the right-to-left direction of the opening 3a is maximum to a further distal end side. FIG. 13 exemplifies a case in which the first blade surface portion 31 and the second blade surface portion 32 are formed in a range from the same position as a distal end 3b of the opening 3a toward the distal end side to a needle tip T in the direction along the axis G.

In the puncture needle 100 of the present embodiment, as illustrated in FIG. 13, a first back surface portion 41 intersects with the first blade surface portion 31 and a third blade surface portion 33. The first back surface portion 41 forms a first side blade edge 51 as a cutting edge by a ridge line formed by intersecting with the first blade surface portion 31 and the third blade surface portion 33.

The first side blade edge 51 includes a distal-end-side first side blade edge 51b formed by a ridge line formed by the first back surface portion 41 and the first blade surface portion 31 intersecting with each other, and a proximal-end-side first side blade edge 51a formed by a ridge line formed by the first back surface portion 41 and the third blade surface portion 33 intersecting with each other. Each of the proximal-end-side first side blade edge 51a and the distal-end-side first side blade edge 51b is formed linearly.

Similarly to the first back surface portion 41, a second back surface portion 42 intersects with the second blade surface portion 32 and the third blade surface portion 33. The second back surface portion 42 forms a second side blade edge 52 as a cutting edge by a ridge line formed by intersecting with the second blade surface portion 32 and the third blade surface portion 33.

The second side blade edge 52 includes a distal-end-side second side blade edge 52b formed by a ridge line formed by the second back surface portion 42 and the second blade surface portion 32 intersecting with each other, and a proximal-end-side second side blade edge 52a formed by a ridge line formed by the second back surface portion 42 and the third blade surface portion 33 intersecting with each other. Each of the proximal-end-side second side blade edge 52a and the distal-end-side second side blade edge 52b is formed linearly.

The proximal-end-side first side blade edge 51a and the proximal-end-side second side blade edge 52a are sharp cutting edges similar to the first side blade edge 51 and the second side blade edge 52 of the first embodiment. The distal-end-side first side blade edge 51b and the distal-end-side second side blade edge 52b are sharp cutting edges similar to the first side blade edge 51 and the second side blade edge 52 of the first embodiment.

Note that, because the first blade surface portion 31 and the second blade surface portion 32 of the front-side blade surface 3 intersect with a lower end surface 40 at the needle tip T to form the distal end blade edge 50, the distal-end-side first side blade edge 51b and the distal-end-side second side blade edge 52b do not intersect with each other at the needle tip T.

### Fourth Embodiment

In the first embodiment described above, the case in which the front-side blade surface 3 is the blade surface that extends from the proximal end side to the distal end side while inclining with respect to the axis G of the main body 1 from the upper side to the lower side is described, and the case in which the front-side blade surface 3 is formed in one planar shape is described. However, the front-side blade surface 3 is not always formed in one planar shape. The front-side blade surface 3 may be a combination of two or more (for example, two) planes.

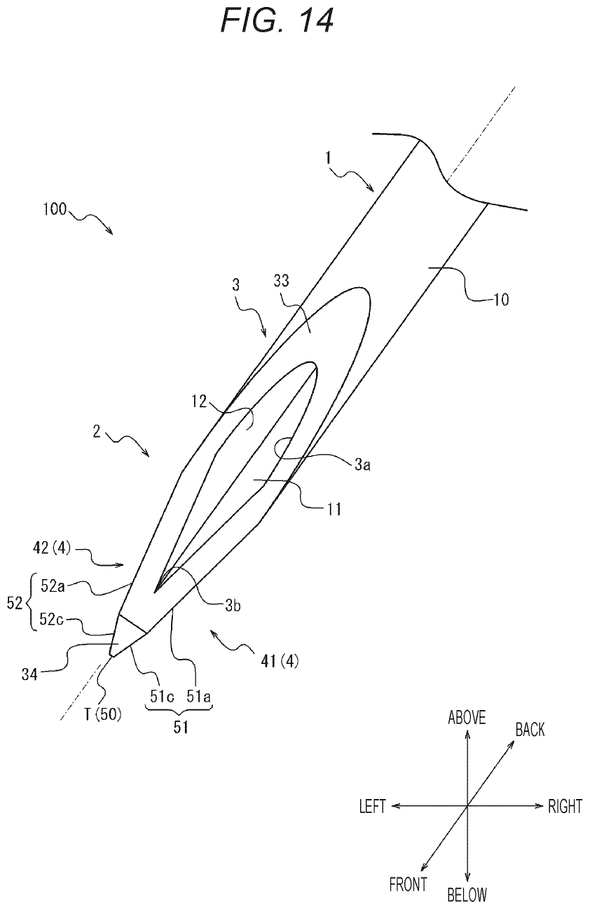

FIGS. 14 to 16 illustrate a puncture needle 100 according to the present embodiment. FIGS. 14 to 16 are a perspective view, a right side view, and a top view of the puncture needle 100, respectively, similarly to FIGS. 1 to 3. The puncture needle 100 according to the present embodiment is different from the puncture needle 100 of the first embodiment with respect to the front-side blade surface 3, but is otherwise the same as the first embodiment. Hereinafter, portions different from those of the first embodiment are mainly described, and the description of the similar portions is omitted as appropriate.

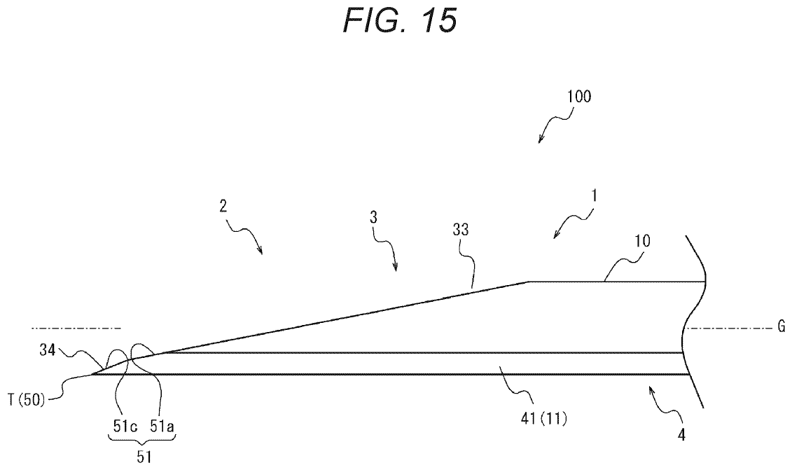

As illustrated in FIG. 14, a blade surface portion 2 of the puncture needle 100 according to the present embodiment includes a third blade surface portion 33 and a fourth blade surface portion 34 as a front-side blade surface 3. As illustrated in FIGS. 14 and 15, the fourth blade surface portion 34 is arranged on a distal end side with respect to the third blade surface portion 33, and is significantly inclined with respect to the axis G as compared with the third blade surface portion 33. Note that, the phrase of “significantly inclined with respect to the axis G as compared with the third blade surface portion 33” means a state closer to a state orthogonal to the axis G than a state closer to parallel to the axis G as compared with the third blade surface portion 33.

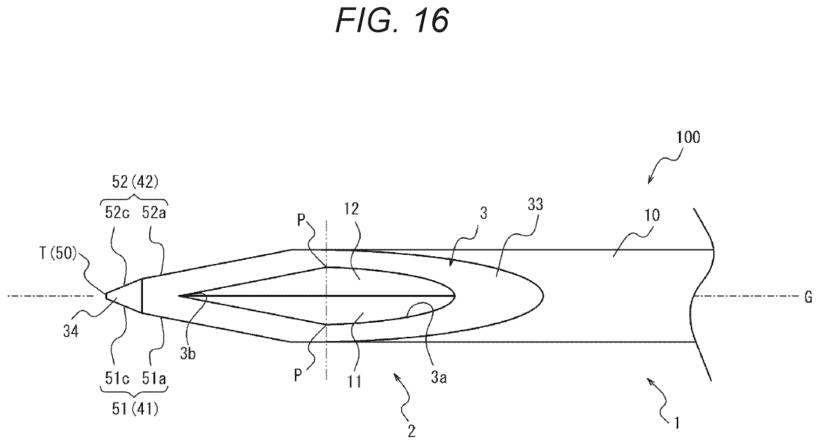

In the present embodiment, as illustrated in FIGS. 14 and 16, a case in which the fourth blade surface portion 34 is formed in a range from a position on the distal end side than the same position as the distal end 3b of the opening 3a toward further distal end side to a needle tip T. A first side blade edge 51 and a second side blade edge 52 extend from the third blade surface portion 33 to the fourth blade surface portion 34.

In the puncture needle 100 of the present embodiment, a first back surface portion 41 intersects with the third blade surface portion 33 and the fourth blade surface portion 34. The first back surface portion 41 forms the first side blade edge 51 as a cutting edge by a ridge line formed by intersecting with the third blade surface portion 33 and the fourth blade surface portion 34.

The first side blade edge 51 includes a distal-end-side first side blade edge 51c formed by a ridge line formed by the first back surface portion 41 and the fourth blade surface portion 34 intersecting with each other, and a proximal-end-side first side blade edge 51a formed by a ridge line formed by the first back surface portion 41 and the third blade surface portion 33 intersecting with each other. Each of the proximal-end-side first side blade edge 51a and the distal-end-side first side blade edge 51c is formed linearly.

Similarly to the first back surface portion 41, a second back surface portion 42 intersects with the third blade surface portion 33 and the fourth blade surface portion 34. The second back surface portion 42 forms a second side blade edge 52 as a cutting edge by a ridge line formed by intersecting with the third blade surface portion 33 and the fourth blade surface portion 34.

The second side blade edge 52 includes a distal-end-side second side blade edge 52c formed by a ridge line formed by the second back surface portion 42 and the fourth blade surface portion 34 intersecting with each other, and a proximal-end-side second side blade edge 52a formed by a ridge line formed by the second back surface portion 42 and the third blade surface portion 33 intersecting with each other. Each of the proximal-end-side second side blade edge 52a and the distal-end-side second side blade edge 52c is formed linearly.

The proximal-end-side first side blade edge 51a and the proximal-end-side second side blade edge 52a are sharp cutting edges similar to the first side blade edge 51 and the second side blade edge 52 of the first embodiment. The distal-end-side first side blade edge 51c and the distal-end-side second side blade edge 52c intersect with the proximal-end-side first side blade edge 51a and the proximal-end-side second side blade edge 52a, but become sharp cutting edges similar to the proximal-end-side first side blade edge 51a and the proximal-end-side second side blade edge 52a.

Note that, because the first blade surface portion 31 and the second blade surface portion 32 of the front-side blade surface 3 intersect with a lower end surface 40 at the needle tip T to form the distal end blade edge 50, the distal-end-side first side blade edge 51c and the distal-end-side second side blade edge 52c do not intersect with each other at the needle tip T.

### Fifth Embodiment

In the first embodiment described above, a case in which the blade surface portion 2 includes the front-side blade surface 3 is described. However, the blade surface portion 2 may further be provided with a back-surface-side blade surface 6 formed on a back surface side of the front-side blade surface 3 in the blade surface portion 2 together with the front-side blade surface 3.

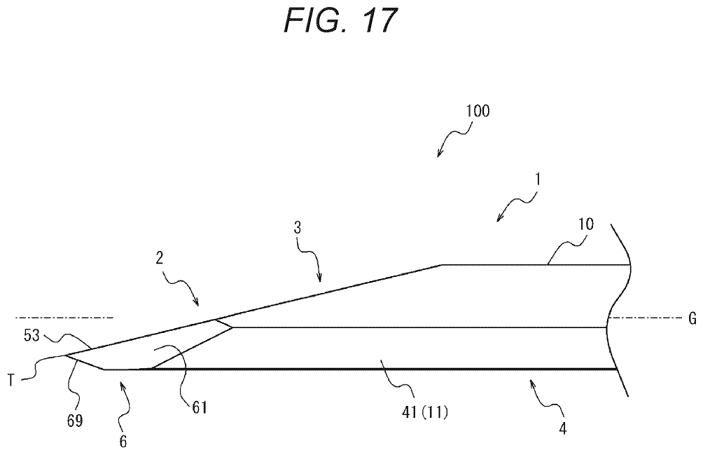

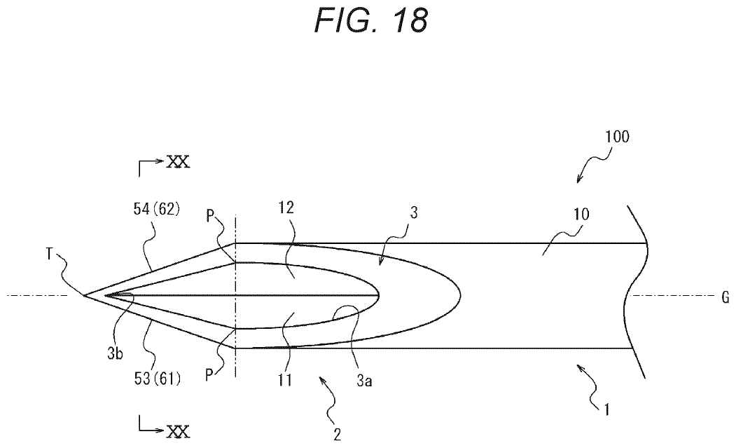

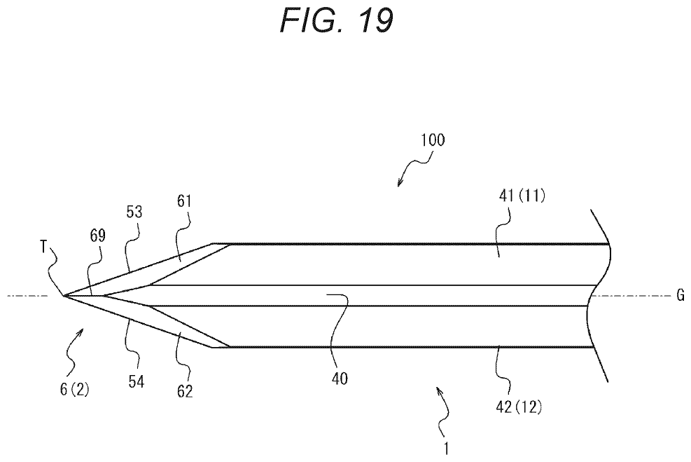

FIGS. 17 to 19 illustrate a puncture needle 100 according to the present embodiment. The puncture needle 100 according to the present embodiment is a so-called back cut type. FIGS. 17 and 18 are a right side view and a top view of the puncture needle 100, respectively, similarly to FIGS. 2 and 3. FIG. 19 is a back view of the puncture needle 100 as seen from a side opposite to FIG. 18. As illustrated in FIGS. 17 to 19, the puncture needle 100 according to the present embodiment is the same as the puncture needle 100 of the first embodiment except that the blade surface portion 2 includes the front-side blade surface 3 and the back-surface-side blade surface 6, and that the first back surface portion 41 and the second back surface portion 42 do not necessarily intersect with the front-side blade surface 3 to form the blade edge. Hereinafter, portions different from those of the first embodiment are mainly described, and the description of the similar portions is omitted as appropriate.

As illustrated in FIG. 19, the back-surface-side blade surface 6 includes a first back-surface-side blade surface portion 61 formed in a planar shape, which intersects with the front-side blade surface 3 (refer to FIGS. 17 and 18) to form a third side blade edge 53 as a cutting edge, and a second back-surface-side blade surface portion 62 formed in a planar shape, which intersects with the front-side blade surface 3 to form a fourth side blade edge 54 as a cutting edge.

The first back-surface-side blade surface portion 61 and the second back-surface-side blade surface portion 62 may be formed from the same position as the position at which the width in the right-to-left direction of the opening 3a in the direction along the axis G is maximum (the position of a virtual line P-P in FIG. 18) or the position slightly closer to the proximal end side toward the distal end side.

The present embodiment illustrates a case in which the first back-surface-side blade surface portion 61 and the second back-surface-side blade surface portion 62 are formed from a position between a position at which the width in the right-to-left direction of the opening 3a in the direction along the axis G is maximum and a distal end 3b of the opening 3a toward the distal end side. A case in which the first back surface portion 41 and the second back surface portion 42 do not intersect with the front-side blade surface 3 is illustrated.

The first back-surface-side blade surface portion 61 and the second back-surface-side blade surface portion 62 intersect with each other at the distal end to form a ridge line, and the ridge line forms a needle tip back-surface-side blade edge 69 including the needle tip T as one end on the back-surface-side blade surface 6. The needle tip back-surface-side blade edge 69 makes a needle tip sharp and improves a puncturing property of the puncture needle 100.

In the puncture needle 100 according to the present embodiment, because the first back surface portion 41 and the second back surface portion 42 are formed on the back surface side of the front-side blade surface 3, it is possible to secure sufficiently long lengths of the third side blade edge 53 and the fourth side blade edge 54 as the cutting edges as compared with a case in which portions corresponding to the first back surface portion 41 and the second back surface portion 42 have a curved surface shape protruding outward as in a case in which the main body 1 is cylindrical. As a result, the puncture needle 100 can secure a necessary and sufficient length of the cutting edge in the blade surface portion 2.

Note that, the first back surface portion 41 and the second back surface portion 42 do not necessarily intersect with the front-side blade surface 3 to form a blade edge, and in the present embodiment, the first back surface portion 41 and the second back surface portion 42 do not intersect with the front-side blade surface 3 to form the blade edge.

Because the main body 1 includes a circular arc-shaped portion 10 having a circular arc-shaped cross section intersecting with the axis G, and planar first plate-shaped portion 11 and second plate-shaped portion 12, the first plate-shaped portion 11 and second plate-shaped portion 12 are arranged on the back surface side, an outer surface of the first plate-shaped portion 11 is the first back surface portion 41, and an outer surface of the second plate-shaped portion 12 is the second back surface portion 42, it is possible to easily avoid an inner diameter interference and secure longer third side blade edge 53 and fourth side blade edge 54 as the cutting edges as compared with a case in which the main body 1 has a cylindrical shape.

A point that the puncture needle 100 can be formed with a sufficiently long cutting edge as compared with the case in which the main body 1 has a cylindrical shape is described in detail below.

As illustrated in FIG. 18, the puncture needle 100 can secure a wide width (distance) between the opening 3a and the third side blade edge 53 and the fourth side blade edge 54 on the front-side blade surface 3 even when the cutting edge is lengthened. That is, in the puncture needle 100, the inner diameter interference in which the opening 3a interferes with the third side blade edge 53 and the fourth side blade edge 54 is easily avoided as compared with a case in which the main body 1 has a cylindrical shape and is back cut. Therefore, the puncture needle 100 can secure a sufficiently long cutting-edge length.

The puncture needle 100 can sufficiently secure a thickness (a thickness between an inner surface of the first plate-shaped portion 11 and the first back-surface-side blade surface portion 61 and a thickness between an inner surface of the second plate-shaped portion 12 and the second back-surface-side blade surface portion 62) of the blade surface portion 2 even when the cutting edge is lengthened. That is, in the puncture needle 100, it is easy to avoid the inner diameter interference in which the inner surface of the tube (the inner surfaces of the first plate-shaped portion 11 and the second plate-shaped portion 12) in the main body 1 and the first back-surface-side blade surface portion 61 and the second back-surface-side blade surface portion 62 interfere with each other, as compared with a case in which the main body 1 has a cylindrical shape and is back cut. Therefore, the puncture needle 100 can secure a sufficiently long cutting-edge length.

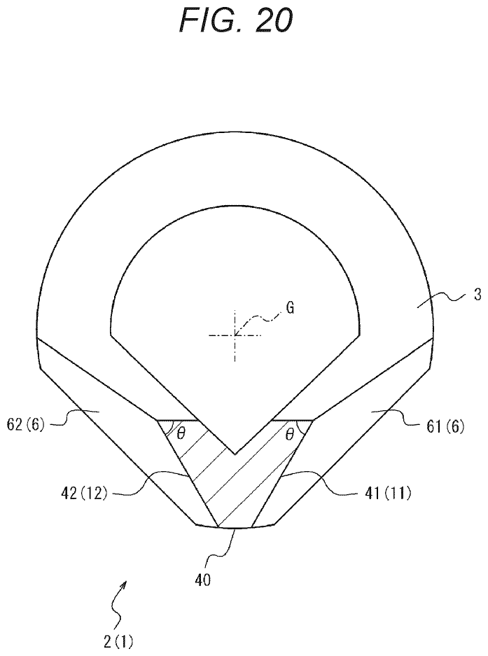

In the puncture needle 100 according to the present embodiment, the lengths of the third side blade edge 53 and the fourth side blade edge 54 can be adjusted (for example, to be longer than a case in which the first back surface portion 41 and the second back surface portion 42 are not formed) while maintaining sharpness (for example, cutting edge angle θ=60° without a blunt cutting edge angle θ (refer to FIG. 20) of the third side blade edge 53 and the fourth side blade edge 54. That is, it is possible to sufficiently secure the length of the cutting edge while keeping the third side blade edge 53 and the fourth side blade edge 54 as cutting edges sharp.

### Sixth Embodiment

In the fifth embodiment described above, the case in which the front-side blade surface 3 is the blade surface that extends from the proximal end side to the distal end side while inclining with respect to the axis G of the main body 1 from the upper side to the lower side is described, and the case in which the front-side blade surface 3 is formed in one planar shape is described. The case in which the blade surface portion 2 is further provided with the back-surface-side blade surface 6 formed on the back surface side of the front-side blade surface 3 in the blade surface portion 2 together with the front-side blade surface 3 is described. However, the front-side blade surface 3 is not always formed in one planar shape. The front-side blade surface 3 may be a combination of two or more (for example, two) planes.

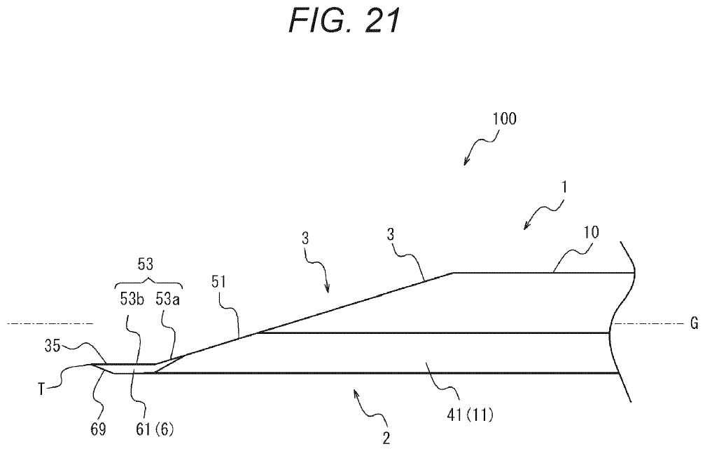

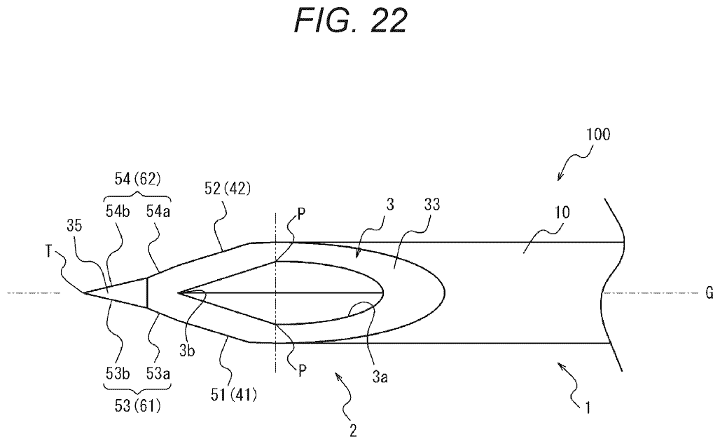

FIGS. 21 and 22 illustrate a puncture needle 100 according to the present embodiment. FIGS. 21 and 22 are a right side view and a top view of the puncture needle 100, respectively, similarly to FIGS. 2 and 3. The puncture needle 100 according to the present embodiment is different from the puncture needle 100 of the fifth embodiment with respect to the front-side blade surface 3, but is otherwise the same as the fifth embodiment. Hereinafter, portions different from those of the fifth embodiment are mainly described, and the description of the similar portions is omitted as appropriate.

The present embodiment illustrates a case in which the back-surface-side blade surface 6 is formed from a position slightly on a proximal end side than a distal end 3b (refer to FIG. 22) of an opening 3a in a direction along an axis G toward the distal end side. A case in which a first back surface portion 41 and a second back surface portion 42 intersect with the front-side blade surface 3 to form a first side blade edge 51 and a second side blade edge 52 is illustrated.

The blade surface portion 2 of the puncture needle 100 according to the present embodiment includes a third blade surface portion 33 and a fifth blade surface portion 35 as the front-side blade surface 3. As illustrated in FIGS. 21 and 22, the fifth blade surface portion 35 is arranged on a distal end side with respect to the third blade surface portion 33, and is gradually inclined with respect to the axis G as compared with the third blade surface portion 33 or parallel to the same. In the present embodiment, a case in which the fifth blade surface portion 35 is parallel to the axis G is described. Note that, the phrase of “gradually inclined with respect to the axis G as compared with the third blade surface portion 33” means a state further closer to the state parallel to the axis G as compared with the third blade surface portion 33, in other words.

In the present embodiment, as illustrated in FIGS. 21 and 22, a case in which the fifth blade surface portion 35 is formed in a range from a position on the distal end side than the same position as the distal end 3b of the opening 3a toward the distal end side to a needle tip T.

In the present embodiment, the first back surface portion 41 and the second back surface portion 42 intersect with the front-side blade surface 3. The first back surface portion 41 and the second back surface portion 42 intersect with the front-side blade surface 3 only at the third blade surface portion 33 to form the first side blade edge 51 and the second side blade edge 52. The first side blade edge 51 and the second side blade edge 52 do not intersect with each other at the needle tip T.

The back-surface-side blade surface 6 intersects with the third blade surface portion 33 and the fifth blade surface portion 35 of the front-side blade surface 3 to form a third side blade edge 53 and a fourth side blade edge 54.

The third side blade edge 53 includes a proximal-end-side third side blade edge 53a and a distal-end-side third side blade edge 53b formed by ridge lines formed by the first back-surface-side blade surface portion 61 of the back-surface-side blade surface 6 intersecting with the third blade surface portion 33 and the fifth blade surface portion 35.

The fourth side blade edge 54 includes a proximal-end-side fourth side blade edge 54a and a distal-end-side fourth side blade edge 54b formed by ridge lines formed by the second back-surface-side blade surface portion 62 of the back-surface-side blade surface 6 intersecting with the third blade surface portion 33 and the fifth blade surface portion 35.

The puncture needle 100 of the present embodiment includes the first side blade edge 51, the second side blade edge 52, the third side blade edge 53, and the fourth side blade edge 54, and includes the cutting edge with a sufficiently secured length as a whole. The first side blade edge 51, the second side blade edge 52, the third side blade edge 53, and the fourth side blade edge 54 are sharp cutting edges similar to the case with the first embodiment and the fifth embodiment.

As described above, it is possible to provide the puncture needle including the sharp cutting edge with a sufficiently secured length.

### Another Embodiment

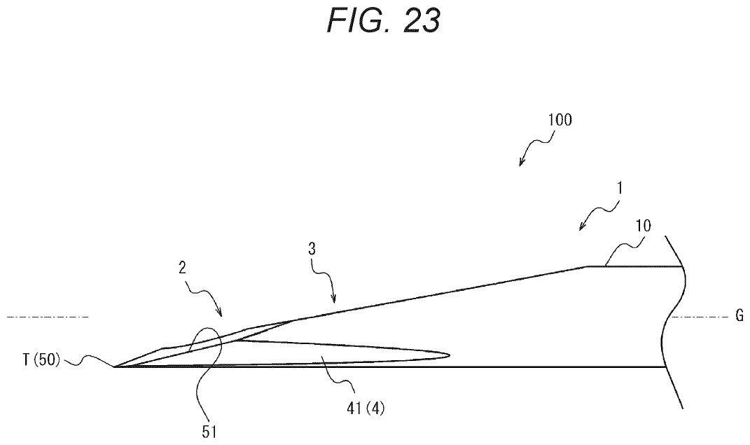

(1) In the above-described embodiment, the case in which the back surface portion 4 extends parallel to the axis G is described, but the back surface portion 4 is not limited to the case of being parallel to the axis G. For example, as illustrated in FIG. 23, the back surface portion 4 (in FIG. 23, a first back surface portion 41) may be formed so as to come closer to the axis G from a proximal end side toward a distal end side. Even in this case, the back surface portion 4 (the first back surface portion 41) does not intersect with other back surface portions (refer to the second back surface portion 42 in FIG. 1, and the like) at a needle tip T.

FIG. 23 illustrates a case in which the first back surface portion 41, which is the back surface portion 4, is formed to extend from a position on the distal end side with respect to a proximal end of a front-side blade surface 3 in a direction along the axis G and on the proximal end side with respect to a position at which a width in a right-to-left direction of an opening 3a in the direction along the axis G is maximum toward a distal end of the main body 1, and further to come closer to the axis G from the proximal end side to the distal end side. Note that, FIG. 23 illustrates a case in which the puncture needle 100 is a lancet type as described in the second embodiment described above.

The main body 1 including such back surface portion 4 can be formed by, for example, performing forming processing of thinning a cylindrical member in a radial direction by grinding, cutting, electrical discharge machining, chemical polishing, and the like. In this case, the forming processing of the main body 1 may be performed after the front-side blade surface 3 is formed. For example, the first back surface portion 41 and the second back surface portion 42 may be formed after the front-side blade surface 3 is formed.

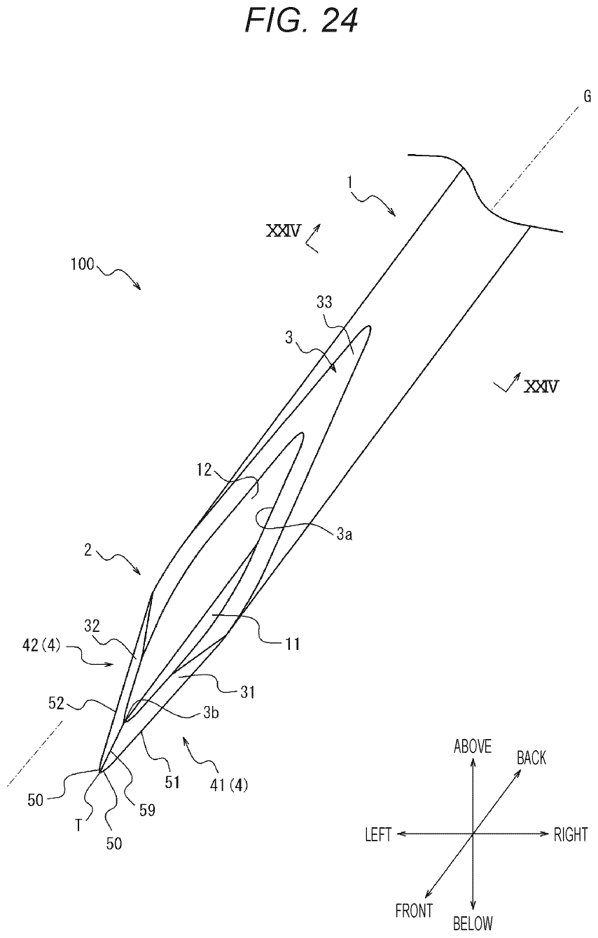

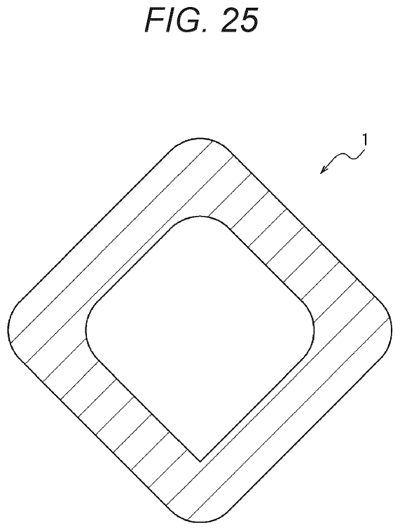

(2) In the above-described embodiment, the case in which the main body 1 includes the circular arc-shaped portion 10 having the circular arc-shaped cross section intersecting with the axis G, and the planar first plate-shaped portion 11 and second plate-shaped portion 12, and the shape of the cross section intersecting with the axis G of the main body 1 is a circular arc shape in which the front surface side is convex upward, and a triangular portion convex downward is added below the circular arc portion is described, but the shape of the main body 1 is not limited to this. As illustrated in FIGS. 24 and 25, the main body 1 may have a rectangular cross section intersecting with the axis G. In a case in which the main body 1 has a rectangular cross section, four corners on the outer surface side may be chamfered. Note that, FIG. 25 is a cross-sectional view taken along line XXV-XXV of FIG. 24. By adopting the rectangular main body 1, it might be easy to form the first plate-shaped portion 11 and the second plate-shaped portion 12 or the back surface portion 4.

The configuration disclosed in the above-described embodiments (including the embodiment described as “another embodiment”, the same applies hereinafter) can be applied in combination with the configuration disclosed in other embodiments as long as there is no contradiction. The embodiments disclosed in the present specification are examples, and the invention is not limited thereto, and can be appropriately modified without departing from the object of the present disclosure.

The present disclosure is applicable to a puncture needle.

What is claimed is:

1. A puncture needle comprising:
a rod-shaped main body;
a blade surface portion formed at a distal end of the main body, the blade surface portion comprising a front-side blade surface extending so as to be inclined with respect to an axis of the main body;
a planar first back surface portion formed on a back surface side of the front-side blade surface at the distal end of the main body; and
a planar second back surface portion formed on the back surface side of the front-side blade surface at the distal end of the main body, wherein:
the first back surface portion and the second back surface portion are parallel to the axis or come closer to each other toward a needle tip, and do not intersect with each other at the needle tip.

2. The puncture needle according to claim 1, wherein:
the first back surface portion intersects with the front-side blade surface to form a first side blade edge,
the second back surface portion intersects with the front-side blade surface to form a second side blade edge, and
the first side blade edge and the second side blade edge come closer to each other toward the needle tip.

3. The puncture needle according to claim 2, wherein the first side blade edge and the second side blade edge are linear.

4. The puncture needle according to claim 2, wherein:
the blade surface portion comprises a first blade surface portion, a second blade surface portion, and a third blade surface portion as the front-side blade surface,
the third blade surface portion is arranged on a proximal end side of the first blade surface portion or the second blade surface portion,
the first blade surface portion and the second blade surface portion intersect with each other to form a needle tip front-side blade edge including the needle tip at one end,
the first back surface portion and the first blade surface portion intersect with each other to form the first side blade edge, and
the second back surface portion and the second blade surface portion intersect with each other to form the second side blade edge.

5. The puncture needle according to claim 4, wherein:
the blade surface portion comprises the third blade surface portion and a fourth blade surface portion as the front-side blade surface,
the fourth blade surface portion is arranged on a distal end side with respect to the third blade surface portion, and
the first side blade edge and the second side blade edge extend from the third blade surface portion to the fourth blade surface portion.

6. The puncture needle according to claim 5, wherein:
an inclination between the fourth blade surface portion and the axis is greater than an inclination between the third blade surface portion and the axis.

7. The puncture needle according to claim 5, wherein:
the fourth blade surface portion is parallel to the axis.

8. The puncture needle according to claim 3, wherein:
the blade surface portion comprises a first blade surface portion, a second blade surface portion, and a third blade surface portion as the front-side blade surface,
the third blade surface portion is arranged on a proximal end side of the first blade surface portion or the second blade surface portion,
the first blade surface portion and the second blade surface portion intersect with each other to form a needle tip front-side blade edge including the needle tip at one end,
the first back surface portion and the first blade surface portion intersect with each other to form the first side blade edge, and
the second back surface portion and the second blade surface portion intersect with each other to form the second side blade edge.

9. The puncture needle according to claim 8, wherein:
the blade surface portion comprises the third blade surface portion and a fourth blade surface portion as the front-side blade surface,
the fourth blade surface portion is arranged on a distal end side with respect to the third blade surface portion, and
the first side blade edge and the second side blade edge extend from the third blade surface portion to the fourth blade surface portion.

10. The puncture needle according to claim 9, wherein:
an inclination between the fourth blade surface portion and the axis is greater than an inclination between the third blade surface portion and the axis.

11. The puncture needle according to claim 9, wherein:
the fourth blade surface portion is parallel to the axis.

12. The puncture needle according to claim 1, further comprising:
a back-surface-side blade surface formed on the back surface side of the front-side blade surface in the blade surface portion, wherein:
the back-surface-side blade surface comprises:
a first back-surface-side blade surface portion formed in a planar shape, the first back-surface-side blade surface portion intersecting with the front-side blade surface to form a third side blade edge, and
a second back-surface-side blade surface portion formed in a planar shape, the second back-surface-side blade surface portion intersecting with the front-side blade surface to form a fourth side blade edge; and
the first back-surface-side blade surface portion and the second back-surface-side blade surface portion intersect with each other to form a needle tip back-surface-side blade edge including the needle tip at one end.

13. The puncture needle according to claim 1, wherein:
the main body is tubular and comprises a circular arc-shaped portion having a circular arc-shaped cross section intersecting with the axis, a first plate-shaped portion and a second plate-shaped portion,
the first back surface portion is an outer surface of the first plate-shaped portion, and
the second back surface portion is an outer surface of the second plate-shaped portion.
